(19) United States
(12) Patent Application Publication
(10) Pub. No.: US 2006/0264827 A1
(43) Pub. Date: Nov. 23, 2006

(54) Secure injector and operating method thereof

(76) Inventor: Michael G. M. Whang, Taipei City (TW)

Correspondence Address: Mr. Joseph A. Sawyer, Jr.;SAWYER LAW GROUP LLP, Embarcadero Corporate Center, 2465 East Bayshore Road, Suite 406, Palo Alto, CA, 94303, US

(21) Appl. No.: 11/221,485

(22) Filed: Sep. 7, 2005

(30) Foreign Application Priority Data
Apr. 29, 2005 (TW) 94113809

(51) Int. Cl. A61M 5/00; A61M 5/32

(52) U.S. Cl. 604/110; 604/195

(57) ABSTRACT

A secure injector and the operating method thereof are proposed. The secure injector includes: a needle cylinder having a body and a needle socket, the body and the needle socket being hollow bodies forming a containing space; a fixing and maintaining section in the form of a hollow tubular body disposed in the needle socket, the fixing and maintaining section including a ringent first fixed section and a second fixed section without puffing; a metallic needle having a withstanding section, the metallic needle being positioned in the hollow tubular body of the fixing and maintaining section, and withstood in the second fixed section by the withstanding section; and an injecting pole having a magnetic component, wherein the magnetic component of the injecting pole attracts the metallic needle through drawing or pulling out the injecting pole, thereby the metallic needle is retracted into the needle cylinder.

### FIELD OF THE INVENTION

The present invention relates generally to the secure injector and operating method thereof. More particularly, the present invention relates to the secure injector in which the needle is automatically retracted into the needle cylinder and an operating method thereof.

### BACKGROUND OF THE INVENTION

It is a common medical procedure to inject medicine into a patient by needle in order to control illness during the treatment process. However, in the process, medical workers are subjected to the risk of being pricked by the injecting needle and receiving an infection. Therefore, medical workers must be extremely cautious when using injection needles in order to avoid being infected.

The Republic of China Laid-Open Patent Publication No.M249673 proposes a retracted-mode secure injector having a telescoping drawhook. This secure injector includes a needle cylinder, a needle, and an injecting pole. The injecting pole is composed of a cork, wherein a handspike body and a drawhook component discretely connect the cork with the handspike body. The drawhook component contains a drawhook formed in front of the cork, wherein the drawhook component and the handspike body move in separate directions a specified distance so as to separate the cork from the handspike body by negative pressure formed in the process of drawing in medicine and also the pull imposed on the injecting pole through the structure of a non-return locking pawl. Moreover, by virtue of the thrust imposed on the injecting pole and the friction of the fringe between the cork and the inner wall of the needle cylinder during the injection process, the cork and the handspike body come back to the jogged state, and part of the drawhook protrudes to the front of the cork. Thus, when the cork contacts the inside of the front of the needle cylinder, the drawhook component connects with the needle, such that by pulling back on the injecting pole, the needle can be withdrawn into the containing space of the needle cylinder.

However, although the above-described conventional retracted-mode secure injector having a telescopic drawhook provides a secure injector capable of retracting the needle to the needle cylinder that avoids the accidental occurrence of medical workers pricking themselves during the process of using the needle cylinder, the structure for such a retracted-mode secure injector having a telescopic drawhook is complex and assembly is not easy. Moreover, the needle is unable to be automatically retracted to the needle cylinder.

Accordingly, there exists a strong need in the art for fabricating a secure injector with simple structure, easy assembly, and a needle that can be automatically retracted into the needle cylinder.

### SUMMARY OF THE INVENTION

Accordingly, it is an objective of the present invention to solve the aforementioned problems by providing a secure injector, wherein the needle can be automatically retracted to the needle cylinder after usage by drawing and pulling out the injecting pole in such a way that the magnetic component set in the injecting pole magnetizes the metallic needle so as to retract the metallic needle to the needle cylinder, thereby avoiding risk to the environment and infection of the hospital staff.

It is another objective of the present invention to provide a secure injector with the needle automatically retracted to the needle cylinder, wherein a magnetic component and hook are set in the injecting pole, and also a spring unit is set in the needle cylinder, such that, when the hook of the injecting pole engages the spring unit, the spring unit is tensioned, and when the injecting pole is released, the spring unit releases, and, concurrently, the magnetic component of the injecting pole magnetizes the metallic needle, thereby the metallic needle can be automatically retracted to the needle cylinder in order to enhance the safety of the hospital staff by insuring that the hospital staff is protected from being pricked by the metallic needle of the injector.

It is further objective of the present invention to provide a secure injector with simple structure and easy assembly which protects the hospital staff from being pricked by the metallic needle of a possibly infectious injector and being infected after finishing the injecting process, thereby realizing the objective of a practical method to ensure safety, so as to maintain the personal safety of the hospital staff.

In order to attain the objectives mentioned above and others, a secure injector according to the present invention comprises: a needle cylinder having a body and a needle socket, the body and the needle socket being hollow structures, wherein a first containing space is sequentially formed in the needle cylinder body and second and third containing spaces are formed in the needle socket, the third containing space extending outside the second containing space; a fixing and maintaining section disposed in the needle socket in the form of hollow tubular body, the fixing and maintaining section including a first fixed section and a second fixed section, the first fixed section being a ringent body; a metallic needle having a withstanding section, the metallic needle being positioned in the hollow tubular body of the fixing and maintaining section, and withstood in the second fixed section of the fixing and maintaining section by the withstanding section; and an injecting pole having a magnetic component, wherein the magnetic component of the injecting pole magnetizes the metallic needle through drawing and pulling out the injecting pole, thereby the metallic needle contained in the fixing and maintaining section is retracted into the needle cylinder.

In addition, the rear of the needle cylinder extends outside and forms a flange on which a spring unit is set, and a plurality of hooks are set at the axial position of the injecting pole. During injecting, when the hook engages the spring unit and the injecting pole is pushed, the spring unit is tensioned, and when the injecting pole is released, the spring unit releases, and, concurrently, the magnetic component of the injecting pole magnetizes the metallic needle, thereby the metallic needle is automatically retracted into the needle cylinder.

In order to attain the objectives mentioned above and others, the present invention is directed to an operating method of the aforementioned secure injector, the method includes the following steps: containing the injection in the needle cylinder through the metallic needle according to the injection dosages as required; pushing the injecting pole to make the end with the magnetic component of the injecting pole extrude the injection, thereby performing the injecting of the injection in the needle cylinder; making the first fixed section of the fixing and maintaining section enter from the second containing space to the third containing space and assume a ringent state, so that the metallic needle assumes a loosened state; and making the magnetic component of the injecting pole attract the metallic needle, drawing and pulling out the injecting pole, so that the metallic is retracted to the needle cylinder.

Furthermore, a hook is set correspondingly at the axial position of the injecting pole and a spring unit is set in the needle cylinder, so that the hook of the injecting pole can engage the spring unit of the needle cylinder, such that, when the injecting pole is pushed for injecting, the spring unit is tensioned towards the injecting pole, and the first fixed section of the fixing and maintaining section enters from the second containing space to the third containing space and assumes a ringent state, thereby the metallic needle assumes a loosened state. Then, when the injecting pole is released, the spring unit retracts to make the injecting pole quickly move in a direction deviating from the first containing space, making the magnetic component of the injecting pole magnetize the metallic needle and move in the drawing and pulling out direction, thereby the metallic needle is automatically retracted into the needle cylinder.

A secure injector and operating method thereof according to the present invention can efficiently solve the conventional problems caused by the complex structure, difficult assembly, and the inability of the needle to be automatically retracted into the needle cylinder of existing designs, and can concurrently protect the hospital staff from being pricked by the needle of an infectious injector and being infected when administering injections.

### BRIEF DESCRIPTION OF THE DRAWINGS

The present invention can be more fully understood by reading the following detailed description of the preferred embodiments, with reference made to the accompanying drawings, wherein:

FIG. 1 is an exploded perspective view of a secure injector according to the present invention;

FIG. 2 is an assembled cross-sectional view of the secure injector according to the present invention;

FIG. 3 is an assembled perspective view of the secure injector according to the present invention;

FIG. 4 is a cross-sectional view of the secure injector when drawing an injection according to the present invention;

FIGS. 5A and 5B are cross-sectional views showing the secure injector safely retracting a metallic needle according to the present invention;

FIG. 6 is a cross-sectional view of the secure injector according to another embodiment of the present invention;

FIG. 7 is a cross-sectional view of the secure injector when drawing the injection according to another embodiment of the present invention;

FIG. 8 is a cross-sectional view of the secure injector during injecting according to another embodiment of the present invention; and

FIGS. 9A and 9B are cross-sectional views showing the secure injector safely retracting the metallic needle according to another embodiment of the present invention.

### DETAILED DESCRIPTION OF THE PREFERRED EMBODIMENTS

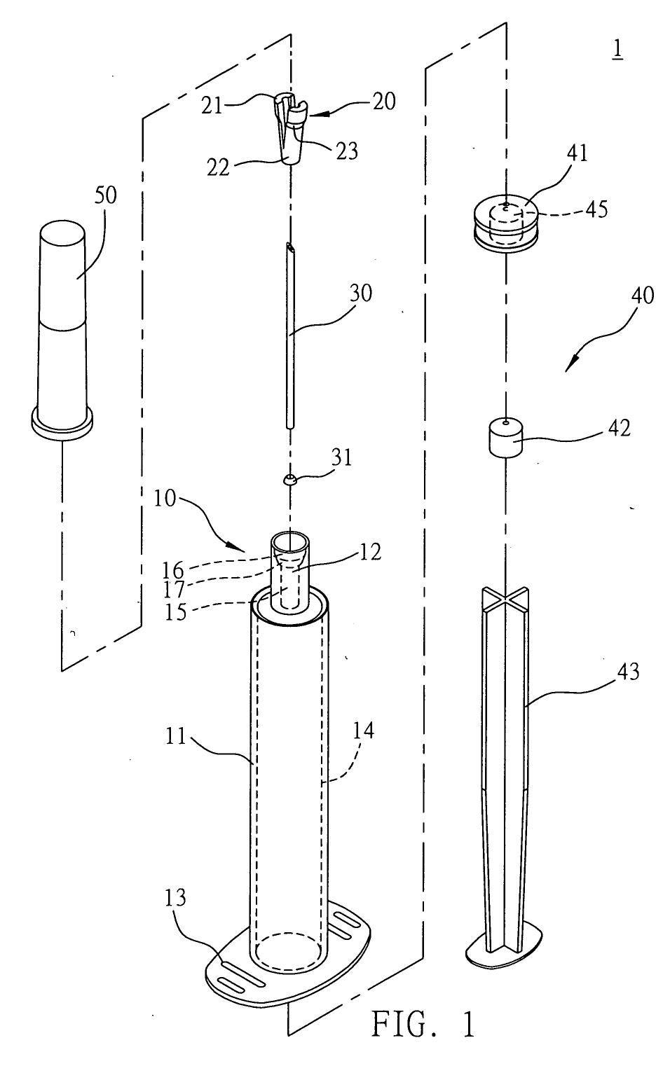

With reference to FIG. 1, an exploded perspective is shown of the secure injector according to the present invention. The secure injector according to the present invention 1 comprises a needle cylinder 10 having a needle cylinder body 11 and a needle socket 12; a fixing and maintaining section 20 having a first fixed section 21 and a second fixed section 22; a metallic needle 30 having a withstanding section 31; and an injecting pole 40 having a cork 41, a magnetic component 42, and an injecting pole body 43.

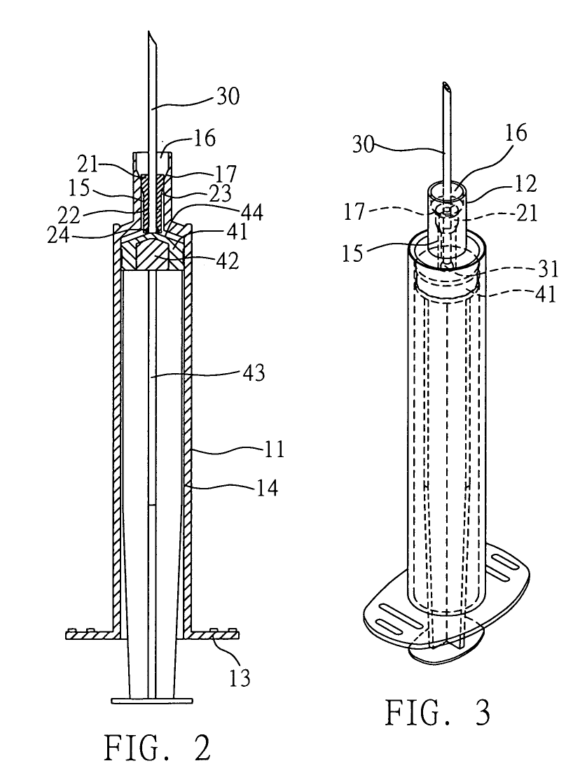

Continuing reference to FIG. 1 and matching with FIG. 2, FIG. 2 depicts a cross section of the secure injector according to the present invention. Two sides of the rear of the needle cylinder 10 extend outside and form a flange 13, the flange 13 is approximately an oval body, while the needle cylinder body 11 is a hollow body, and the hollow body forms a first containing space 14, the first containing space 14 of the needle cylinder body 11 being for receiving the injecting pole 40.

The needle socket 12 is also a hollow body connecting with the needle cylinder body. The hollow body of the needle socket 12 sequentially forms a second containing space 15 and a third containing space 16, the third containing space 16 extending outside from the second containing space 15, so that the inner diameter of the third containing space 16 is larger than that of the second containing space 15. The second containing space 15 and the third containing space 16 of the needle socket 12 are for placing the fixing and maintaining section 20, and a beveled portion 17 is set between the second containing space 15 and the third containing space 16, the third containing space 16 being larger than the second containing space 15 due to the beveled portion 17. The inside of the needle socket 12 forms a shape that is wide at the top and narrow at the bottom, and the beveled portion 17 is to lead the first fixed section 21 of the fixing and maintaining section 20 into the third containing space 16 in the needle socket 12 of the needle cylinder 10.

The fixing and maintaining section 20 is a hollow body for placing the metallic needle 30. In its natural state, the first fixed section 21 of the fixing and maintaining section 20 is ringent in shape without the effect of applying force. There is a beveled portion 23 between the first fixed section 21 and the second fixed section 22. The external diameter of the first fixed section 21 of the fixing and maintaining section extends outside from the second fixed section 22 along the beveled portion 23, such that the external diameter of the first fixed section 21 is larger than the inner diameter of the second containing space 15 in the needle socket 12, the external diameter of the second fixed section 22 is smaller than the inner diameter of the second containing space 15 in the needle socket 12, thus, the fixing and maintaining section 20 can be closely contained in the second containing space 15 of the needle socket 12 by the first fixed section 21, and the first fixed section 21 assumes a closed state due to the application of external force. In addition, the beveled portion 23 of the fixing and maintaining section 20 is matched with the beveled portion 17 between the second containing space 15 and the third containing space 16. As such, the first fixed section 21 of the fixing and maintaining section 20 easily enters the third containing space 16 under light pressure. The inner diameter of the third containing space 16 is larger than the external diameter of the first fixed section 21, such that the first fixed section 21 can not be pressed any more by the external force and can assume a ringent state when the first fixed section 21 enters the third containing space 16. Furthermore, the external diameter of the metallic needle 30 is slightly larger than the inner diameter of the hollow tube of the fixing and maintaining section 20. The fixing and maintaining section 20 and the needle cylinder 10 can employ plastic material having the plasticity, thereby the metallic needle 30 can be closely gripped in the fixing and maintaining section 20 contained in the needle socket 12 (the second containing space 15). Concurrently, the possibility that injection could filter into the inside of the fixing and maintaining section 20 or the interval between the fixing and maintaining section 20 and the needle socket 12 is greatly reduced or avoided.

A flute 24 for propping the withstanding section 31 of the metallic needle 30 is set at the bottom of the second fixed section 22 of the fixing and maintaining section 20 so that the metallic needle 30 can not slide forward. In addition, the second fixed section 22 can slightly protrude into the first containing space 14 in the needle cylinder body 11 so that the injecting pole 40 can efficiently push the fixing and maintaining section 20 during the process of injecting, and the fixing and maintaining section 20 can move so as to make the first fixed section enter the third containing space 16 in the needle socket 12 and assume a ringent state.

The injecting pole 40 comprises: a cork 41 at one end having a containing space 45, a magnetic component 42, the magnetic component 42 being positioned in the containing space 45 of the cork 41, and an injecting pole body 43, the injecting pole body 43 being an axial, cross-shaped rib, but not limited to this design. In addition, the cork 41 at one end of the injecting pole 40 can be formed with a slight bulge 44, so as to efficiently push the fixing and maintaining section 20 gripped in the second containing space 15 of the needle socket 12.

Furthermore, the secure injector further comprises a cover body 50 for combining with the needle socket 12, so as to cover the metallic needle 30.

In a preferred, exemplary embodiment, the metallic needle 30 and the withstanding section 31 are integrally molded, meaning that the rear of the metallic needle 30 extends outward and forms a withstanding section 31 in a one-piece design. The formation of the injecting pole 40 consists of pouring the cork 41 having magnetic material, and then pouring the injecting pole body 43 from plastic material in a pouring mold to form the injecting pole 40 having the magnetic component according to the present invention.

With reference to FIG. 3, shown is a perspective view of the secure injector according to the present invention. In the secure injector 1 of the present invention, the metallic needle 30 is positioned in the hollow tubular body of the fixing and maintaining section 20 through the rear of the fixing and maintaining section 20. At this time, the withstanding section 31 of the metallic needle 30 butts up against the flute 24 of the fixing and maintaining section 20, so that the metallic needle can not slide forward and can be disposed in the fixing and maintaining section 20. The fixing and maintaining section 20 is positioned in the second containing space 15 of the needle socket 12, and the rear of the fixing and maintaining section 20 protrudes into the needle cylinder body 11. The first fixed section 21 of the fixing and maintaining section 20 originally assuming a ringent state is closely combined with the needle socket 12 and firmly disposed in the needle socket 12, and the metallic needle 30 is also firmly disposed in the fixing and maintaining section 20 and can not slide. The magnetic component 42 is positioned in the cork 41 and then combined with the injecting pole body 43, and the assembled injecting pole 40 is inserted from the rear of the needle cylinder body 11 and disposed in the first containing space 14.

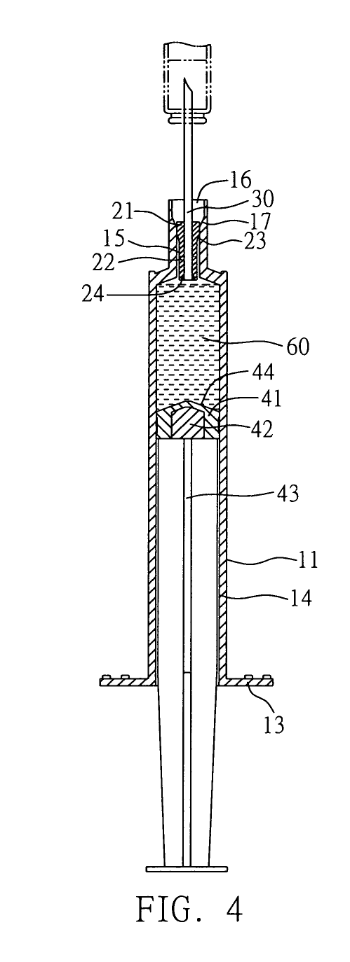

With reference to FIG. 4, a cross-sectional view is shown of the secure injector when drawing in the injection according to the present invention. When it is desired to draw in the injection 60 using the secure injector 1 of the present invention, the injecting pole 40 is progressively pulled in the direction away from the needle socket 12. This pulling action forms a negative pressure in the needle cylinder 10, so that the injection 60 is drawn into the needle cylinder 10. At this time, the fixing and maintaining section 20 is still positioned in the second containing space 15 of the needle socket 12, and the metallic needle 30 is firmly positioned in the fixing and maintaining section 20 by virtue of the gripping of the fixing and maintaining section 20. And when the needle cylinder 10 has the desired amount of injection 60 desired to perform the injection, the injection is administered by slowly pushing the injecting pole 40 progressively into the first containing space 14 until the injecting pole 40 is firmly pushed against the fixing and maintaining section 20, thereby completing the action of injecting. At this time, the fixing and maintaining section 20 is still in the second containing space 15 of the needle socket 12, and the metallic needle 30 is firmly positioned in the fixing and maintaining section 20 by virtue of the gripping of the fixing and maintaining section 20.

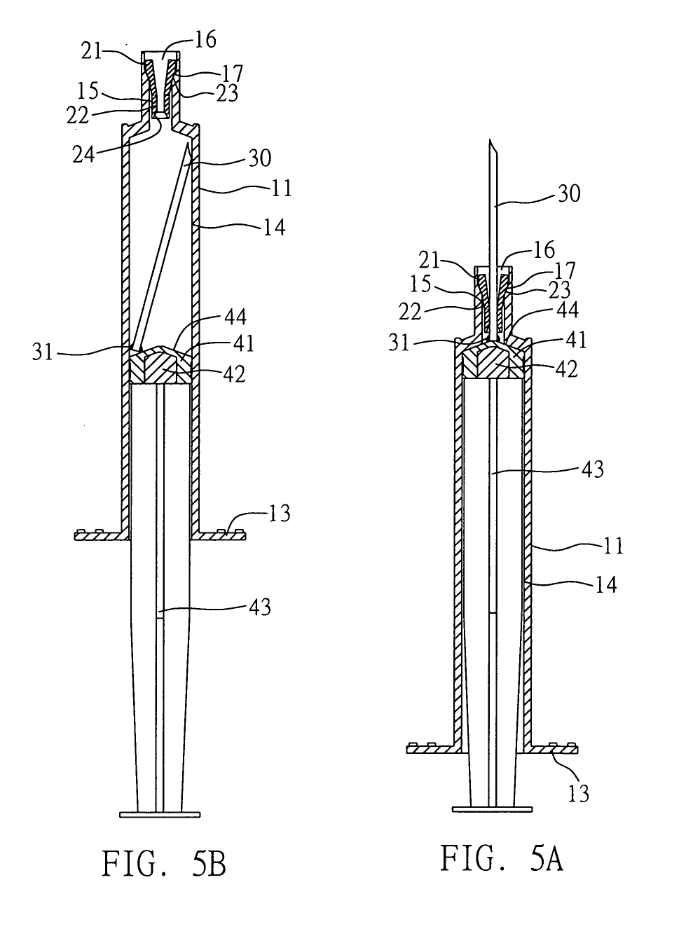

With reference to FIGS. 5A and 5B, cross-sectional views are shown of the secure injector showing the process of safely retracting the metallic needle according to the present invention in order to avoid the injector from posing a risk of pricking someone. When injecting is finished, applying pressure on the injecting pole 40 firmly abuts it against the bottom of the fixing and maintaining section 20. This causes the first fixed section 21 of the fixing and maintaining section 20 to be pushed firmly into the third containing space 16 having a relative large space. The bulge 44 of the injecting pole 40 helps to push the fixing and maintaining section 20. After being pushed sufficiently, the first fixed section 21 assumes a ringent state, and the metallic needle 30 then assumes a loosened state due to the opening of the first fixed section 21. Then, through drawing out the injecting pole 40, the magnetic component 42 of the injecting pole 40 attracts the metallic needle 30 and makes the metallic needle 30 move in the drawing and pulling out direction, thereby the metallic needle 30 is contained in the needle cylinder 10, attaining the feature of a secure injector.

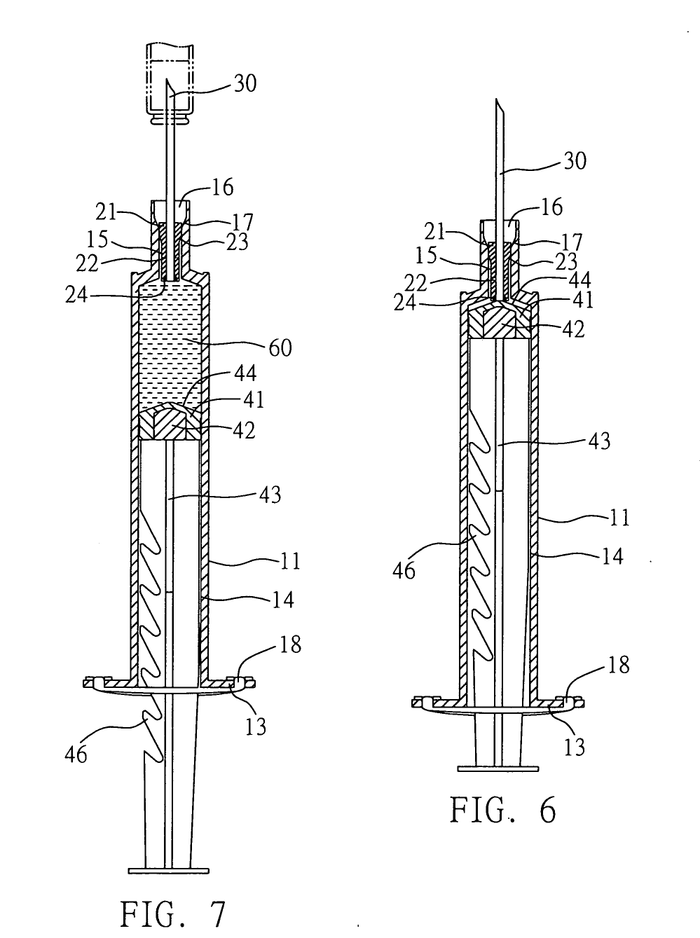

With reference to FIG. 6, a cross-sectional view is shown of the secure injector according to another embodiment of the present invention. Components that are identical or similar to the above exemplary embodiment are given the same or similar component reference numerals. For conciseness, detailed descriptions for components identical to the above embodiment are omitted.

The secure injector according to the present invention comprises a needle cylinder 10 having a needle cylinder body 11 and a needle socket 12. A spring unit 18 is set at the rear of the needle cylinder 10. The secure injector further comprises: a fixing and maintaining section 20 having a first fixed section 21 and a second fixed section 22; a metallic needle 30 having a withstanding section 31; and a injecting pole 40 having a cork 41, a magnetic component 42, an injecting pole body 43, and a plurality of hooks 46 axially set along the injecting pole body.

Two sides of the rear of the needle cylinder 10 extend outside and form a flange 13, and the spring unit 18 is set on the flange 13. The needle cylinder body 11 is a hollow body, and the hollow body forms a first containing space 14, the first containing space 14 of the needle cylinder body 11 being for placing the injecting pole 40 and containing the injection 60.

The needle socket 12 is also a hollow body forming a second containing space 15 and a third containing space 16, the second containing space 15 and the third containing space 16 of the needle socket 12 being for placing the fixing and maintaining section 20. There is a beveled portion 17 between the second containing space 15 and the third containing space 16, the third containing space 16 being larger than the second containing space 15 due to the beveled portion 17. The inside of the needle socket 12 forms a shape that is wide at the top and narrow at the bottom.

The fixing and maintaining section 20 has a hollow tube for placing the metallic needle 30. A beveled portion 23 matching with the beveled portion 17 between the second containing space 15 and the third containing space 16 is also set between the first fixed section 21 and the second fixed section 22, so that the first fixed section 21 of the fixing and maintaining section 20 easily enters the third containing space 16 of the needle socket 12, and, in its natural state, the first fixed section 21 of the fixing and maintaining section 20 is ringent in shape. A flute 24 for propping the withstanding section 31 of the metallic needle 30 is set at the bottom of the second fixed section 22, so that the metallic needle 30 can not slide forward. In addition, the beveled portion 17 of the aforementioned needle socket 12 is used to lead the first fixed section 21 of the fixing and maintaining section 20 into the third containing space 16 of the needle socket 12 and make the first fixed section 21 assume a ringent state. The withstanding section 31 at one end of the metallic needle 30 withstands in the second fixed section 22 of the fixing and maintaining section.

The external diameter of the first fixed section 21 is larger than the inner diameter of the second containing space 15 in the needle socket 12. The external diameter of the second fixed section 22 is smaller than the inner diameter of the second containing space 15 in the needle socket 12, thus, the fixing and maintaining section 20 can be closely contained in the second containing space 15 of the needle socket 12 by the first fixed section 21, and the first fixed section 21 assumes a closed state due to the application of external force. In addition, the beveled portion 23 of the fixing and maintaining section 20 is matched with the beveled portion 17 between the second containing space 15 and the third containing space 16, thereby the first fixed section 21 of the fixing and maintaining section 20 can be pushed easily to enter the third containing space 16. The inner diameter of the third containing space 16 is larger than the external diameter of the first fixed section 21, so that the first fixed section 21 can not be pressed further by external force and can assume a ringent state when the first fixed section 21 enters the third containing space 16. Furthermore, the external diameter of the metallic needle 30 is slightly larger than the inner diameter of the hollow tube of the fixing and maintaining section 20. The fixing and maintaining section 20 and the needle cylinder 10 can employ plastic material having plasticity, thereby the metallic needle 30 can be closely gripped in the fixing and maintaining section 20 contained in the needle socket 12 (the second containing space 15), and, concurrently, injection filtering into the inside of the fixing and maintaining section 20 or the interval between the fixing and maintaining section 20 and the needle socket 12 is greatly reduced or avoided.

The injecting pole 40 comprises: a cork 41 positioned at the front end having a containing space 45, a magnetic component 42 formed in the containing space 45 of the cork 41, and an axial injecting pole body 43 that is cross-shaped, but it is not limited to this. In addition, the cork 41 at one end of the injecting pole 40 can be formed with a slight bulge 44, so as to better push the fixing and maintaining section 20 gripped in the second containing space 15 of the needle socket 12.

Furthermore, a plurality of hooks 46 is set at one side of the injecting pole 40 (on one of the axial, cross-shaped ribs). The distribution of the plurality of the hooks 46 is according to the various injection dosages typically used for injections. That is, the plurality of hooks 46 are set at positions with different interval lengths, e.g., according to the scale of 5 cc, 10 cc, 15 cc, 20 cc etc. in the needle cylinder. Thus, the most rearward hook of the plurality of hooks 46 positioned on one of the axial, cross-shaped ribs corresponds to an injection dosage of 5 cc; the next hook corresponds to an injection dosage of 10 cc; and so forth. The hooks 46 are used to engage the spring unit 18. In addition, the design of the hooks 46 assumes a barbed, oblique shape, such that, when the injecting pole 40 moves towards the rear opening of the needle cylinder during the process of drawing in an injection, the hooks can not engage the spring unit 18 and prevent the injecting pole 40 from moving. Correspondingly, when the injecting pole 40 moves towards the needle socket 12 during the process of injecting an injection into the recipient, the injecting pole 40 engages the spring unit 18 by the hooks 46 to tension the spring. Subsequently, the injecting pole 40 is automatically retracted by the stored energy in the spring unit 18 when injecting is finished and the injection pole 40 is released by the user administering the injection.

Continuing reference to FIG. 6 and matching with FIG. 7, FIG. 7 depicts a cross-sectional view of the secure injector when drawing the injection according to another embodiment of the present invention. When it is desired to draw in an injection 60 using the secure injector 1 of this embodiment, firstly, drawing out the injecting pole 40 away from the needle socket 12 creates a negative pressure in the needle cylinder 10, such that the injection 60 is drawn into the needle cylinder 10. At this time, the fixing and maintaining section 20 is still positioned in the second containing space 15 of the needle socket 12, and the metallic needle 30 is firmly positioned in the fixing and maintaining section 20 by virtue of the gripping of the fixing and maintaining section 20.

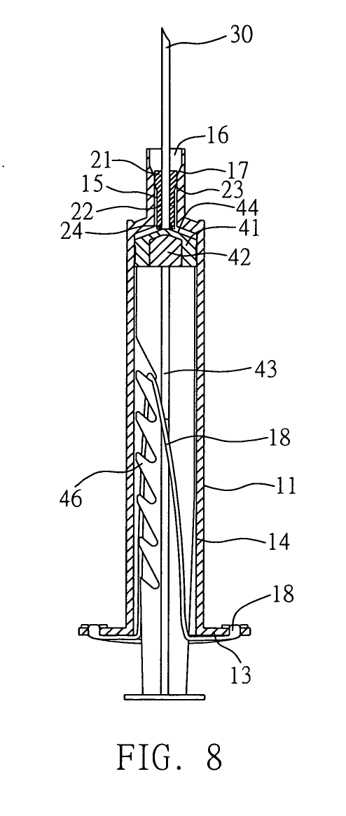

With reference to FIG. 8, a cross-sectional view is shown of the secure injector at the moment of injecting according to another embodiment of the present invention. When it is desired to perform an injection with the needle cylinder 10 filled with an injection 60, the angle of the injecting pole 40 is adjusted, so that one side having a hook 46 of the injecting pole 40 vertically crosses with the flange 13, such that the position of the hook 46 is selected according to the desired injection dosage, and the selected hook 46 of the injecting pole 40 hitches the spring unit 18 of the flange 13. At this time, if the hook 46 of the injecting pole 40 is set to correspond to the stack shell of the needle cylinder, the hook 40 can engage the spring unit 18. Then, the injecting pole 40 is pushed to administer the injection, typically until completely finishing injecting the injection 60, during which the spring unit 18 tensions the spring due to the pressure of pushing until the injecting pole 40 is firmly pushed to the bottom of the fixing and maintaining section 20, thereby completing the action of injecting. At this time, the fixing and maintaining section 20 is still in the second containing space 15 of the needle socket 12, and the metallic needle 30 is firmly positioned in the fixing and maintaining section 20 by virtue of the gripping of the fixing and maintaining section 20.

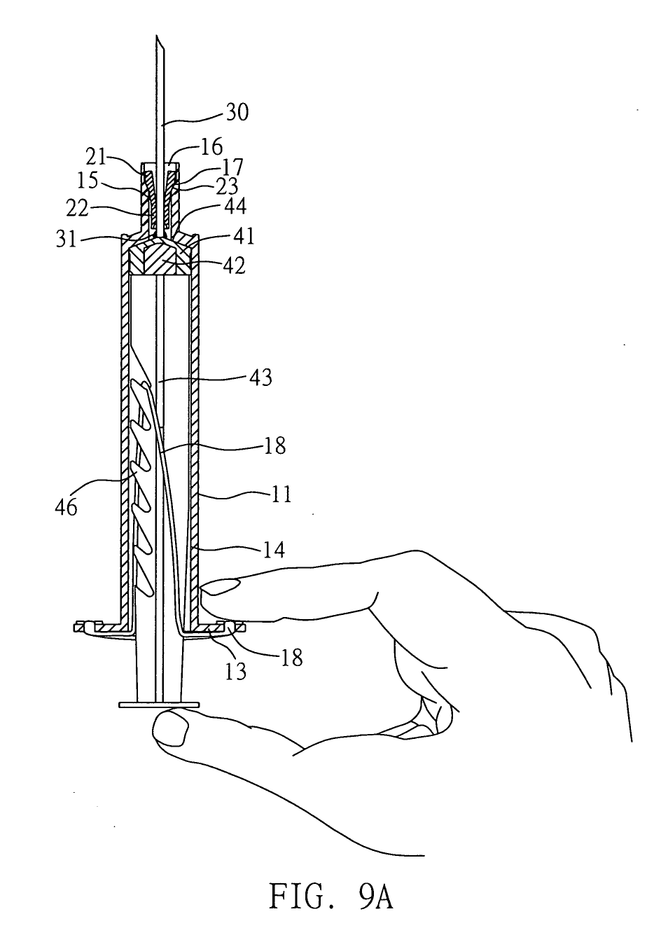

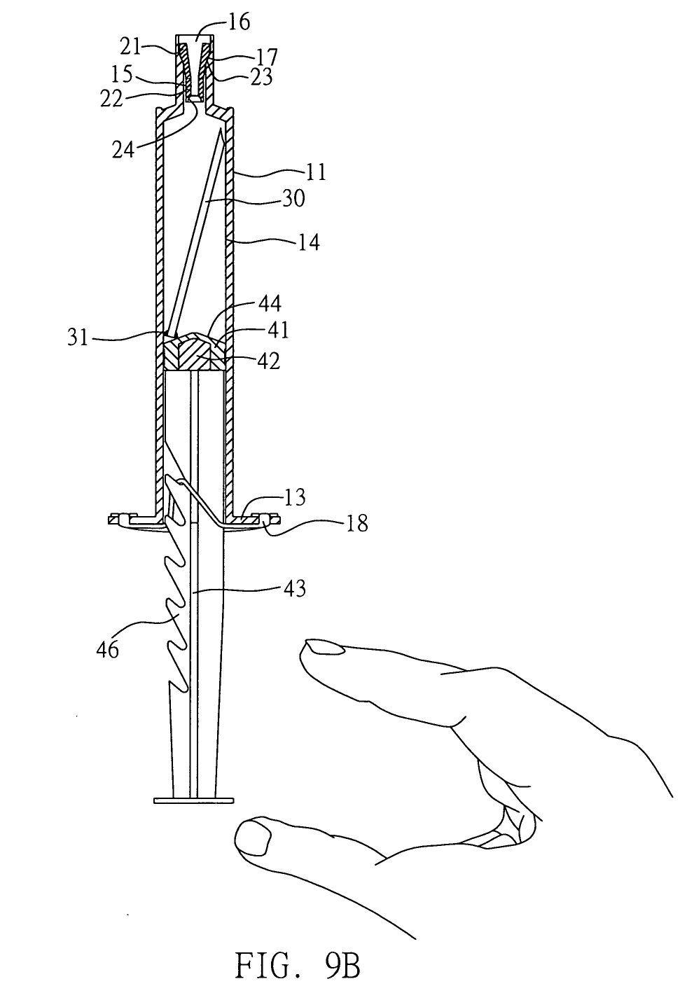

With reference to FIGS. 9A and 9B, cross-sectional views are shown of the secure injector safely retracting the metallic needle according to another embodiment of the present invention in order to avoid the needle from being exposed and possibly pricking someone and causing infection. At the time that the injector finishes the injection, pressure is being applied to the injecting pole 40 to firmly press it the bottom of the fixing and maintaining section 20. The first fixed section 21 of the fixing and maintaining section 20 is pushed to the third containing space 16 with a relatively large space such that the bulge 44 of the injecting pole 40 withstands and pushes the fixing and maintaining section 20 or because that the fixing and maintaining section 20 slightly protrudes to the inside of the needle cylinder, and the first fixed section 21 can not be constrained any more by the external force and can assume a ringent state. At this time, the metallic needle 30 assumes a loosened state due to the opening of the first fixed section 21, while the injector can unloosen by the finger pushing the injecting pole 40, so that the spring unit 18 is released and retracts to make the injecting pole 40 flick towards the direction deviating from the first containing space 14. The magnetic component 42 of the injecting pole 40 concurrently magnetizes and attracts the metallic needle 30, and the metallic needle 30 can move in the drawing out direction of the injecting pole 40, thereby containing the metallic needle 30 in the needle cylinder, and attaining the feature of secure injector with the metallic needle 30 automatically retracted to the needle cylinder 10.

The present invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

What is claimed is:

1. A secure injector comprising:
a needle cylinder having a needle cylinder body and a needle socket, the needle cylinder body and the needle socket being hollow bodies, wherein a first containing space is formed in the needle cylinder body, and a second containing space and a third containing space are formed in the needle socket, the third containing space being extended outside from the second containing space;
a fixing and maintaining section in the form of a hollow tubular body disposed in the needle socket, the fixing and maintaining section including a first fixed section and a second fixed section, wherein the first fixed section is capable of being in a ringent state or in a closed state;
a metallic needle having a withstanding section, the metallic needle being positioned closely in the hollow tubular body of the fixing and maintaining section and withstood in the second fixed section of the fixing and maintaining section by the withstanding section; and
an injecting pole disposed in the needle cylinder, the injecting pole having a body and a magnetic component at one end thereof, wherein the metallic needle is attracted by the magnetic component of the injecting pole through drawing/pulling of the body of the injecting pole so as to retract the metallic needle into the needle cylinder.

2. The secure injector of claim 1, wherein the rear of the needle cylinder is extended outside to form a flange.

3. The secure injector of claim 1, wherein a beveled portion is provided between the second containing space and the third containing space, for allowing the third containing space to extend outside from the second containing space, and for leading the first fixed section of the fixing and maintaining section into the third containing space of the needle socket to be in the ringent state.

4. The secure injector of claim 1, wherein a beveled portion is provided between the first fixed section and the second fixed section of the fixing and maintaining section, so as to allow the first fixed section to enter the third containing space of the needle socket to be in the ringent state.

5. The secure injector of claim 1, wherein the second fixed section of the fixing and maintaining section is slightly projected into the first containing space of the needle cylinder body, so as to allow the first fixed section of the fixing and maintaining section to enter the third containing space of the needle socket to be in the ringent state when the fixing and maintaining section is pushed by the injecting pole.

6. The secure injector of claim 1, wherein the injecting pole further comprises a bulge, so as to allow the first fixed section of the fixing and maintaining section to enter the third containing space of the needle socket to be in the ringent state when the fixing and maintaining section disposed in the needle socket is pushed by the bulge of the injecting pole.

7. The secure injector of claim 1, wherein a flute is provided at the bottom of the second fixed section, for propping the withstanding section of the metallic needle to prevent forward sliding of the metallic needle.

8. The secure injector of claim 1, wherein the metallic needle and the withstanding section are integrally formed.

9. The secure injector of claim 1, wherein the end of the injecting pole is provided with a cork having a containing space for placing the magnetic component therein.

10. The secure injector of claim 1, wherein magnetic material is poured at the end of the injecting pole to form the magnetic component and plastic material is poured to form the body of the injecting pole in a pouring mold.

11. The secure injector of claim 1, further comprising a spring unit disposed in an opening of the first containing space of the needle cylinder.

12. The secure injector of claim 2, further comprising a spring unit disposed on the flange.

13. The secure injector of claim 1, further comprising a plurality of hooks axially provided on the injecting pole.

14. The secure injector of claim 13, wherein the plurality of hooks are arranged according to a scale of injection dosage for injecting.

15. The secure injector of claim 13, wherein the hooks have a barbed and oblique-angled shape, such that when the injecting pole is moved towards an opening of the needle cylinder, the hooks are prevented from engaging the spring unit and interfering with the movement of the injecting pole, and when the injecting pole is moved away from the opening of the needle cylinder, the injecting pole is allowed to engage the spring unit by the hooks.

16. The secure injector of claim 1, further comprising:
a spring unit disposed in an opening of the first containing space of the needle cylinder; and
a plurality of hooks axially provided on the injecting pole and for engaging the spring unit, such that when the injecting pole is pushed, the spring unit is tensioned, and when the injecting pole is released, the spring unit retracts and the metallic needle is attracted by the magnetic component of the injecting pole to retract the metallic needle into the needle cylinder.

17. The secure injector of claim 1, wherein an external diameter of the metallic needle is larger than an inner diameter of the hollow tubular body of the fixing and maintaining section, and wherein the first fixed section of the fixing and maintaining section is extended outside from the second fixed section along a beveled portion provided between the first and second fixed sections such that an external diameter of the first fixed section is larger than an inner diameter of the second containing space of the needle socket, and an external diameter of the second fixed section is smaller than the inner diameter of the second containing space of the needle socket, such that the fixing and maintaining section is closely disposed in the second containing space of the needle socket by the first fixed section, and the first fixed section is in the closed state due to application of external force, so as to allow the metallic needle to be gripped in the fixing and maintaining section disposed in the second containing space of the needle socket, the fixing and maintaining section and the needle cylinder being made of plastic material having plasticity.

18. An operating method of a secure injector comprising the steps of:
providing an injection in a needle cylinder by drawing the injection with a metallic needle according to a predetermined injection dosage;
pushing an injecting pole to allow the end with a magnetic component thereof to extrude the injection so as to perform injecting of the injection in the needle cylinder;
allowing the first fixed section of the fixing and maintaining section to enter the third containing space to be in a ringent state so as to have the metallic needle in a loosened state; and
allowing the magnetic component of the injecting pole to attract the metallic needle, and drawing/pulling the injecting pole to retract the metallic needle into the needle cylinder.

19. The operating method of claim 18, wherein the rear of the needle cylinder extends outside to form a flange.

20. The operating method of claim 18, wherein the secure injector further comprises a spring unit disposed at an opening of the first containing space of the needle cylinder, and a plurality of hooks axially provided on the injecting pole to engage the spring unit, such that when the injecting pole is pushed, the spring unit is tensioned, and when the injecting pole is released, the spring unit retracts and the metallic needle is attracted by the magnetic component of the injecting pole to retract the metallic needle into the needle cylinder.

21. The operating method of claim 20, wherein the plurality of hooks are arranged according to a scale of the injection dosage for injecting of the injection.

22. The operating method of claim 20, wherein the hooks have a barbed and oblique shape such that when the injecting pole is moved towards an opening of the needle cylinder, the hooks are prevented from engaging the spring unit and interfering with the movement of the injecting pole, and when the injecting pole is moved away from the opening of the needle cylinder, the injecting pole is allowed to engage the spring unit by the hooks.

23. The operating method of claim 18, wherein an external diameter of the metallic needle is larger than an inner diameter of the hollow tubular body of the fixing and maintaining section, and wherein the first fixed section of the fixing and maintaining section is extended outside from the second fixed section along a beveled portion provided between the first and second fixed sections such that an external diameter of the first fixed section is larger than an inner diameter of the second containing space of the needle socket, and an external diameter of the second fixed section is smaller than the inner diameter of the second containing space of the needle socket, such that the fixing and maintaining section is closely disposed in the second containing space of the needle socket by the first fixed section, and the first fixed section is in the closed state due to application of external force, so as to allow the metallic needle to be gripped in the fixing and maintaining section disposed in the second containing space of the needle socket, the fixing and maintaining section and the needle cylinder being made of plastic material having plasticity.
